(19) United States
(12) Patent Application Publication
(10) Pub. No.: US 2011/0225705 A1
(43) Pub. Date: Sep. 22, 2011

(54) HEARING PROTECTIVE DEVICE WITH MOISTURE RESISTANT EARMUFF SOUND ABSORBERS

(75) Inventors: RAFAEL P. FERNANDES, CAMPINAS (BR); ELEM CRISTINA CARLOS RIBEIRO, SUMARE (BR); RODRIGO H. MAZZA, CAMPINAS (BR); EMILIO CARLOS P. DE GOUVEA, CURITIBA (BR)

(73) Assignee: 3M Innovative Properties Company

(21) Appl. No.: 12/724,535

(22) Filed: Mar. 16, 2010

(51) Int. Cl. A42B 1/06; A61F 11/00; B23P 17/04

(52) U.S. Cl. 2/209; 128/866; 29/428

(57) ABSTRACT

An earmuff hearing protective device whose earmuffs each include a generally cup-shaped, substantially rigid shell having a head-facing rim; a head-engaging, ear-encircling cushion affixed to the rim; and a sound absorber inside the shell. The sound absorber has a moisture-resistant integral skin substantially surrounding an open cell foam interior having lower density and greater porosity than that of the skin. The skin helps discourage moisture pickup and microbial growth.

### FIELD OF THE INVENTION

This invention relates to earmuff-style hearing protective devices.

### BACKGROUND

Earmuff-style hearing protective devices are widely used in industry for protecting workers against environmental noise. Typically such devices include a pair of cup-shaped rigid shells fastened to a headband, helmet or other headpiece. Each shell typically includes a cushion to improve comfort and to seal the shell against the side of a wearer's head. The shell interior typically includes a sound absorber or liner whose main function is noise blocking. The sound absorber may be cut from a flat sheet or molded to fit the shell. In order to provide optimal sound absorption, the sound absorber usually is made from open cell foam.

### SUMMARY OF THE INVENTION

Conventional open cell earmuff sound absorbers can absorb not only sound but also water, sweat and condensed moisture. This can be a particular problem for wearers engaged in heavy labor in cold humid environments, owing to perspiration emanating from the wearer which may condense within the sound absorber. In order to remove such condensation or other moisture, the sound absorber has to be dried periodically and in some cases removed and replaced. If this is not done, microorganisms may proliferate within the sound absorber, leading to undesirable odors or other complications.

The present invention provides, in one aspect, an earmuff hearing protective device comprising:
a) a headpiece;
b) a pair of earmuffs affixed to the headpiece, each earmuff comprising:
   i. a generally cup-shaped, substantially rigid shell having a head-facing rim;
   ii. a head-engaging, ear-encircling cushion affixed to the rim; and
   iii. a sound absorber inside the shell, the sound absorber having a moisture-resistant integral skin substantially surrounding an open cell foam interior having lower density and greater porosity than that of the skin.

The invention provides, in another aspect, a sound absorber for use in a hearing protective device having an earmuff comprising a generally cup-shaped, substantially rigid shell having a head-facing rim and a head-engaging, ear-encircling cushion affixed to the rim, the sound absorber having a moisture-resistant integral skin substantially surrounding an open cell foam interior having lower density and greater porosity than that of the skin and being shaped to fit inside and line the shell.

The invention provides, in another aspect, a method for making an earmuff hearing protective device, which method comprises:
a) providing a headpiece;
b) providing a pair of earmuffs comprising a generally cup-shaped, substantially rigid shell having a head-facing rim and a head-engaging, ear-encircling cushion affixed to the rim;
c) placing a sound absorber inside each shell, the sound absorber having a moisture-resistant integral skin substantially surrounding an open cell foam interior having lower density and greater porosity than that of the skin; and
d) affixing the earmuffs to the headpiece.

The moisture-resistant integral skin helps to discourage moisture absorption, condensation and soiling within or on the sound absorber.

These and other aspects of the invention will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWING

FIG. 1 is a plan view of an earmuff hearing protective device whose earmuffs are affixed to a headband;

FIG. 2 is a plan view of an earmuff hearing protective device whose earmuffs are affixed to a helmet;

FIG. 3 is an exploded plan view of an earmuff showing its shell, sound absorber and cushion;

FIG. 4 is a three quarter perspective view of the sound absorber of FIG. 3; and

FIG. 5 is a cross-sectional view of the sound absorber of FIG. 3 and FIG. 4.

Like reference symbols in the various figures of the drawing indicate like elements. The elements in the drawings are not to scale.

### DETAILED DESCRIPTION

The term “earmuff” means a component that is dimensioned to encircle an ear of a wearer and is constructed to provide sound attenuation.

The term “headband” means a device that is constructed to retain an earmuff and compress an earmuff against the head of a user.

The phrase “hearing protective device” refers to a personal device, also referred to a as hearing protector, worn to reduce harmful auditory or annoying subjective effects of sound.

The term “integral” when used with respect to a skin portion of an earmuff sound absorber means that the skin is not readily separable from the remainder of the sound absorber.

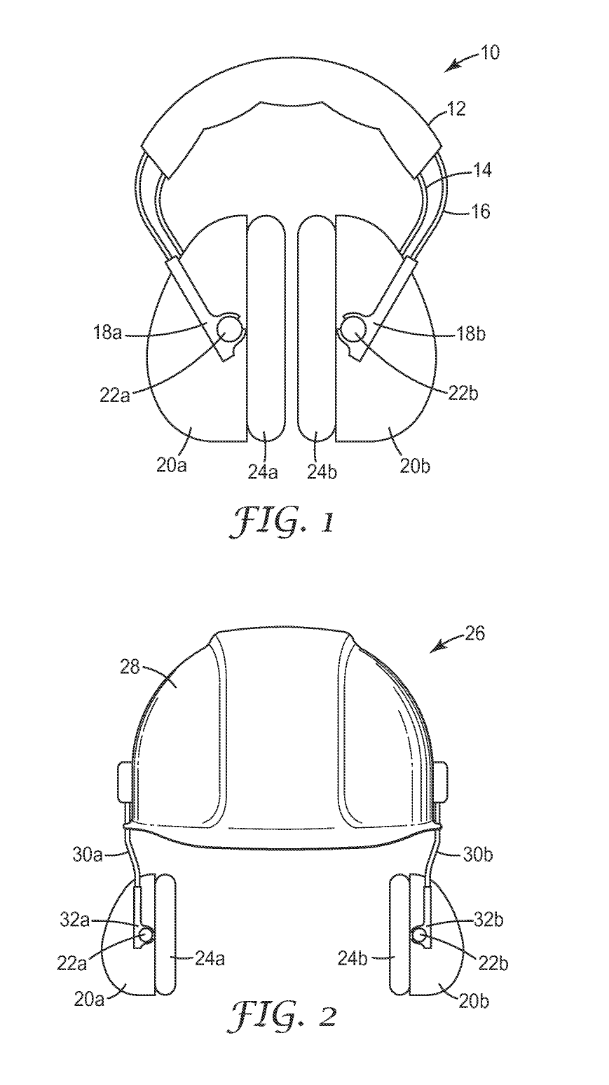

Referring to FIG. 1, hearing protective device 10 includes a headpiece in the form of a headband 12 through which run two inwardly-biased wire supports 14 and 16. The ends of wire support 16 terminate in molded earmuff attachment clips 18a and 18b, and the ends of wire support 14 terminate in two additional molded earmuff attachment clips not shown in FIG. 1. Attachment clips 18a and 18b respectively pivotably affix earmuff shells 20a and 20b to headband 12 by partially capturing pivot pins 22a and 22b. Ear-encircling cushions 24a and 24b are respectively affixed to the rims of shells 20a and 20b, and provide a comfortable fit and sound-attenuating seal when protective device 10 is worn by a wearer.

FIG. 2 shows hearing protective device 26 which includes a headpiece in the form of a helmet 28. Inwardly-biased wire supports 30a and 30b are respectively affixed to the sides of helmet 28, and terminate in molded earmuff attachment clips 32a and 32b. Helmet 28 may include two additional wire supports and attachment clips not shown in FIG. 2. Attachment clips 32a and 32b respectively pivotably affix earmuff shells 20a and 20b to helmet 28 by partially capturing pivot pins 22a and 22b. Earmuff shells 20a and 20b also include ear-encircling cushions 24a and 24b as in FIG. 1.

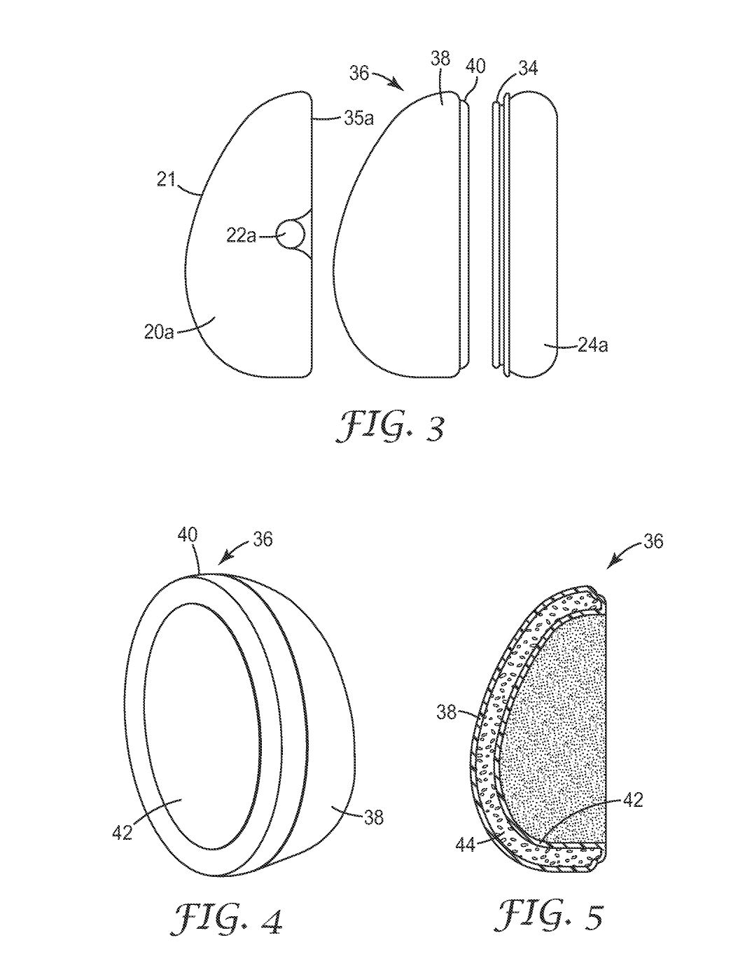

FIG. 3 shows an exploded view of earmuff shell 20a, sound absorber 38 and cushion 24a. Earmuff shell 20a has a cup-shaped distal exterior surface 21 with a generally arcuate convex shape, a rim 35a and a concave interior surface proximate the wearer's ear. Shell 20a may be made from a variety of materials that will be familiar to persons having ordinary skill in the art, such as styrene, various polyolefins, polycarbonates or other impact resistant plastics. Cushion 24a includes a locking bead 34 which engages a mating recess (not shown in FIG. 3) just inside the rim 35a of shell 20a. Cushion 24a may for example have a composite construction with a generally impervious outer cover and a resilient interior, and may be made from a variety of materials that will be familiar to persons having ordinary skill in the art, such as the rubber-like cover material and slow recovery polyurethane foam interior described in U.S. Pat. No. 5,996,123 (Light et al.). Rim 35a and mating cushion 24a may have any suitable shape including, e.g., circular, oval, elliptical, round, square or rectangular, and can define an aperture having any suitable shape including, e.g., circular, oval, elliptical, round, square or rectangular. Accordingly, it will be understood that if cushion 24a is said to be “ear-encircling” or a “ring”, that does not mean that cushion 24a is necessarily circular or any other round form. Sound absorber 36 has an integral exterior skin portion 38 discussed in more detail below. Sound absorber 36 is shaped and dimensioned to fit inside shell 20a, and may include a generally conical surface 40 which is shaped and dimensioned to provide an effective acoustic seal against cushion 24a.

FIG. 4 shows a perspective view of sound absorber 36. Sound absorber 36 has an inner recess with an integral skin portion 42 also discussed in more detail below. The inner recess desirably is shaped and dimensioned to provide good sound attenuation while avoiding contact with the external meatus of a wearer's ear.

FIG. 5 shows a cross-sectional view of sound absorber 36. The interior 44 of sound absorber 36 is a porous, open cell foam having lower density and greater porosity than integral skin portions 38 and 42. Skin portions 38 and 42 may be similar or different, and may have reduced porosity or even no porosity compared to interior 44. Together, skin portions 38 and 42 substantially and preferably completely surround interior 44. If desired, the exterior of sound absorber 36 may include small openings or other regions whose porosity is equal to or greater than that of interior 44, e.g., to assist in drainage or to aid in compressing sound absorber 36 prior to insertion in shell 20a. Integral skin portions 38 and 42 inhibit or prevent the transmission of water, perspiration or other forms of moisture into the interior 44 of sound absorber 36, thereby reducing one or more of condensation, saturation or microbial growth inside sound absorber 36. Integral skin portions 38 and 42 also may help sound absorber 36 resist dirt and soiling, and may make it easier to clean sound absorber 36 if it becomes soiled.

The disclosed hearing protective device desirably provides an average (mean) attenuation of at least 3 and preferably more than 6 decibels when tested according to one or more of ANSI S12.6-1997 Method B, ANSI S3.19-1974, or Section 4.2 of EN 13819-2: 2002.

The disclosed sound absorber may be made from a variety of sound-attenuating or sound-absorbing gas-filled cellular materials. The chosen sound absorber material desirably exhibits appropriate mechanical properties and formability (e.g., by molding, cutting, shaping or a combination thereof) so that the sound absorber may be inserted in (and from time to time as need be removed from and reinstalled) in the earmuff shell. Depending in part on the shape and elasticity of the earmuff shell and cushion, the sound absorber may be a hard foam, semi-rigid foam, or flexible foam, and if resilient may be a slow recovery or instantaneous recovery foam. Exemplary polymers from which the sound absorber may be formed include, e.g., polyurethanes, polyvinyl chloride, and combinations thereof. The foam may be made with a blowing agent or other additive providing reduced thermal conductivity, as doing so may assist in reducing thermal transmission through and condensation within the sound absorber. Exemplary blowing agents include water, chlorofluorocarbons, methylene chloride, acetone, liquid carbon dioxide, formic acid and derivatives such as methyl formate. The ingredients from which the foam is made may include one or more surfactants, catalysts, bactericides, mildewcides, UV inhibitors and other adjuvants. The sound absorber preferably is formed by injection molding using a closed mold and low injection pressure, and formation of the integral skin layer preferably is encouraged by appropriately controlling (e.g., chilling) one or more of the mold walls. Representative foam materials and manufacturing techniques which may be adapted for use in the present invention include those described in U.S. Pat. Nos. 3,644,168 (Bonk et al.), 3,816,233 (Powers), 3,824,199 (Nadeau et al.), 5,266,234 (Ho et al.), 5,476,619 (Nakamura et al.), 5,979,451 (Light et al.), 5,996,123 (Light et al.) and 7,444,687 B2 (Sato et al.).

The completed sound absorber preferably has foam cells with a relatively larger average cross-sectional area and lower density at the interior of the sound absorber, and a relatively smaller average cross-sectional area and higher density at the integral skin surfaces, e.g., at surfaces 38 and 42. The sound absorber may have a range of average foam density values, for example an average density of about 100 to about 1100 kg/m³, and preferably about 150 to about 220 kg/m³. The integral skin surface or surfaces may be open cell or closed cell, may have uniform or varying thickness, porosity or water permeability, and may have an average thickness of for example about 0.2 mm to about 4 mm. The sound absorber and its skin desirably are dimensioned and constructed to discourage or reduce condensation on or the absorption of perspiration and other moisture into sound absorber 36, while still attenuating or absorbing sufficient sound to enable the associated device to qualify as a hearing protective device. For example, condensation may tend to occur on the inside of the earmuff shell, especially at regions in which the sound absorber contacts the shell. The sound absorber skin helps discourage absorption by the sound absorber of condensation occurring near such contact regions. The sound absorber preferably remains free of condensation, perspiration and other moisture even when used under cold humid conditions such as temperatures less than about 20° C. and relative humidity greater than about 50%.

The sound absorber optionally may include a coloring agent or indicia, e.g., one or more dyes, pigments or combinations thereof. The chosen coloring agent or indicia may provide identification, a desired aesthetic property, a visible indication of the sound attenuation properties provided by the sound absorber or device, or combinations thereof. Such coloring agents or indicia may also or instead be incorporated in or on the earmuff shell or other portions of the disclosed device.

Additional details concerning the disclosed device including construction of the headpiece, the provision of size adjustments, the use of alternative measures for affixing the earmuffs to the headpiece, the addition of a retaining strap and other features or alterations will be familiar to persons having ordinary skill in the art and may be found, for example, in the above-mentioned U.S. Pat. Nos. 5,979,451, 5,996,123 and 7,444,687 B2.

The invention is further illustrated in the following examples, in which all parts, percentages and ratios are by weight unless otherwise indicated.

### Example 1

One of the open cell polyurethane flat cut foam sheet sound absorbers in a PELTOR™ Model H9A OPTIME™ 98 Over-the-Head Earmuff (from Aearo Company) was replaced with a polyurethane sound absorber made using low pressure injection molding and a mold whose walls were maintained at 40 to 60° C. The resulting sound absorber had a smooth, glossy exterior skin with a thickness of about 2±0.5 mm and an open cell porous interior with large visible cells. The sound absorber in an additional Model H9A earmuff was replaced with the molded non-skinned sound absorber from a 3M™ Model 1435 Earmuff Hearing Protective Device. The original and both modified earmuffs were suspended open side down about 20 cm above a beaker of water so that they touched one another and were all at the same height above the beaker. To simulate a cold environment, the earmuffs were each covered with a plastic bag containing ice. Using a hot plate, the water was heated to a temperature of about 65° C. for about 45 minutes. Meanwhile, the temperature of the earmuff cup surfaces remained at about 12° C. The sound absorbers were removed from the earmuffs and the sound absorbers and shells were inspected. No condensation was observed inside the skinned molded sound absorber or its shell. Condensation was observed in the other two sound absorbers and their shells, with the most condensation being observed for the Model H9A flat cut foam sheet sound absorber and somewhat less condensation being observed for the Model 1435 non-skinned molded sound absorber. Weight measurements indicated that the moisture content in the skinned molded sound absorber was approximately 62% less than that of the flat cut sheet, whereas the moisture content in the non-skinned molded sound absorber was approximately 41% less than that of the flat cut sheet.

The earmuffs were submitted to a comparative test to evaluate attenuation using a mechanical head and noise generator, and the average Noise Reduction Rate (NRR) was calculated for each earmuff according to ANSI Standard 53.19-1974. The attenuation results are shown below in Table 1, and demonstrate that the skinned molded sound absorber provided attenuation comparable to that provided by the other two sound absorbers:

TABLE 1
NRR Results

| Sound absorber | NRR Average, dB |
|---|---|
| Model H9A OPTIME 98 Foam Sheet | 26 |
| Absorber | |
| Model 1435 Molded Sound absorber | 27 |
| Skinned Molded Sound absorber | 26 |

All references cited herein are expressly incorporated herein by reference in their entirety into this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure and the claims shown below are not limited to the illustrative embodiments set forth herein.

We claim:

1. An earmuff hearing protective device comprising:
a) a headpiece;
b) a pair of earmuffs affixed to the headpiece, each earmuff comprising:
i. a generally cup-shaped, substantially rigid shell having a head-facing rim;
ii. a head-engaging, ear-encircling cushion affixed to the rim:
iii. a sound absorber inside the shell, the sound absorber having a moisture-resistant integral skin substantially surrounding an open cell foam interior having lower density and greater porosity than that of the skin.

2. A device according to claim 1 wherein the headpiece comprises a headband.

3. A device according to claim 1 wherein the headpiece comprises a helmet.

4. A device according to claim 1 wherein the foam comprises flexible foam.

5. A device according to claim 1 wherein the foam comprises polyurethane foam.

6. A device according to claim 1 wherein the sound absorber has an average density of about 100 to about 1100 kg/m³.

7. A device according to claim 1 wherein the sound absorber has an average density of about 150 to about 220 kg/m³.

8. A device according to claim 1 wherein the sound absorber further comprises bactericide or mildewcide.

9. A device according to claim 1 wherein the skin completely surrounds the interior.

10. A device according to claim 1 wherein the skin is porous.

11. A device according to claim 1 wherein the skin is non-porous.

12. A device according to claim 1 wherein the skin has an average thickness of about 0.2 mm to about 4 mm.

13. A device according to claim 1 wherein the foam comprises a blowing agent that imparts reduced thermal conductivity to the sound absorber.

14. A device according to claim 1 wherein the sound absorber remains free of condensed water even when the device is worn at a temperature less than about 20° C. and relative humidity greater than about 50 percent.

15. A sound absorber for use in a hearing protective device having an earmuff comprising a generally cup-shaped, substantially rigid shell having a head-facing rim and a head-engaging, ear-encircling cushion affixed to the rim, the sound absorber having a moisture-resistant integral skin substantially surrounding an open cell foam interior having lower density and greater porosity than that of the skin and being shaped to fit inside and line the shell.

16. A sound absorber according to claim 15 wherein the foam comprises flexible foam.

17. A sound absorber according to claim 15 wherein the foam comprises polyurethane foam.

18. A sound absorber according to claim 15 having an average density of about 100 to about 1100 kg/m³.

19. A sound absorber according to claim 15 having an average density of about 150 to about 220 kg/m³.

20. A sound absorber according to claim 15 further comprising bactericide or mildewcide.

21. A sound absorber according to claim 15 wherein the skin completely surrounds the interior.

22. A sound absorber according to claim 15 wherein the skin is porous.

23. A sound absorber according to claim 15 wherein the skin is non-porous.

24. A sound absorber according to claim 15 wherein the skin has an average thickness of about 0.2 mm to about 4 mm.

25. A sound absorber according to claim 15 wherein the foam comprises a blowing agent that imparts reduced thermal conductivity to the sound absorber.

26. A method for making an earmuff hearing protective device, which method comprises:
a) providing a headpiece
b) providing a pair of earmuffs comprising a generally cup-shaped, substantially rigid shell having a head-facing rim and a head-engaging, ear-encircling cushion affixed to the rim;
c) placing a sound absorber inside each shell, the sound absorber having a moisture-resistant integral skin substantially surrounding an open cell foam interior having lower density and greater porosity than that of the skin; and
d) affixing the earmuffs to the headpiece.
